(19) United States
(12) Patent Application Publication
(10) Pub. No.: US 2017/0265755 A1
(43) Pub. Date: Sep. 21, 2017

(54) IGLASS: INFRARED THERMOGRAPHY FOR LEARNING THERMOREGULATION PERFORMANCE

(71) Applicants: Burcin Becerik-Gerber, Los Angeles, CA (US); Guillermo Castro, Los Angeles, CA (US); Ali Ghahramani, Los Angeles, CA (US)

(72) Inventors: Burcin Becerik-Gerber, Los Angeles, CA (US); Guillermo Castro, Los Angeles, CA (US); Ali Ghahramani, Los Angeles, CA (US)

(73) Assignee: UNIVERSITY OF SOUTHERN CALIFORNIA, Los Angeles, CA (US)

(21) Appl. No.: 15/403,599

(22) Filed: Jan. 11, 2017

Related U.S. Application Data
(60) Provisional application No. 62/277,043, filed on Jan. 11, 2016.

(51) Int. Cl. A61B 5/02; G06N 99/00; A61B 5/16; A61B 5/00; A61B 5/01; A61B 5/0205

(52) U.S. Cl. CPC A61B 5/02007; A61B 5/015; A61B 5/02055; A61B 5/16; A61B 5/7246; G06N 99/005

(57) ABSTRACT

An infrared thermography based technique is described for monitoring an individual's thermoregulation performance and thermal comfort level through measuring the skin temperature on several points the face as the face has a high density of blood vessels and it is typically not covered by clothing. This technique allows for continuous monitoring during normal daily activities and instantaneous identification of thermoregulation performance and thermal comfort. The vascular territories in addition to vasodilation and vasoconstriction of the blood vessels can be used to estimate personal thermal comfort levels. Systems for implementing the technique are described, and can include one or more infrared sensors implemented on glasses for detecting temperature. Data from the sensors is processed by a suitable processor and memory. The processor can continuously monitor the person's blood vessels shrinking and widening which represents thermoregulation performance. Uncomfortable/harmful conditions can be detected by monitoring trends in measurements before the conditions actually occur.

### CROSS-REFERENCE TO RELATED APPLICATION

The present application claims the benefit of U.S. Provisional Application No. 62/277,043, filed on Jan. 11, 2016, which is hereby incorporated by reference in its entirety for all purposes.

### STATEMENT REGARDING FEDERALLY SPONSORED RESEARCH

This invention was made with government support under Contract No. 1351701, awarded by the National Science Foundation (NSF) and under Contract No. 45000176833, awarded by the United States Department of Education (DOE). The Government has certain rights in the invention.

### BACKGROUND

Understanding human thermal comfort (human's perception of the thermal environment) and human body thermoregulation system performance plays an important role in various fields of medicine, physiology, and air conditioning systems. In medical sciences, having access to above thermoregulation system performance information helps the diagnostics of diseases, monitoring the health status of patients, and validating the effectiveness of current prescriptions and practice. The current technique to obtain the required information is to use contact (oral, axillary, and rectal) thermometers which cannot be used for creating streams of the patient's sensing over long period. In physiology domain, monitoring the body temperature helps better understanding how human body thermoregulation is performing under different conditions and activities for the purpose of providing conditions that maximizes the human body health and performance. One of most utilized practice in the physiology domain is to use wearable sensors that are attached to certain body points (often around chest and hands). However, due to the intrusions that it causes to the end users, it could not gain market acceptability and its application is still very limited. In air conditioning system control domain, the objective is to provide satisfactory thermal conditions for the building occupants. Acceptable thermal conditions can be defined with respect to (1) perceptions of the environment, and (2) physiological measurements of thermoregulation system responses. In standards, the air conditioning systems are required to satisfy the perceptions (which is commonly called comfort). The current practice in the industry is to use a thermostat readings located somewhere in a room as a feedback signal in the single variable control loop of the air conditioning systems. However, there are significant variations in air temperature variations across a room ventilated through few duct openings located generally on room ceilings. In addition, significant variations in terms of thermal preferences have been observed among individuals and lack of information about the variations results in conservative HVAC operational settings. It is interesting to note that it had been shown that 7 to 15% of HVAC related energy consumption could be saved by increasing the temperature set point by 1° C. in warm seasons in three large cities (i.e., San Francisco, Phoenix and Miami) in the United States [5]. Considering the fact that Commercial and residential buildings in the United States consume about 40% of the total energy (43% of which is consumed by air conditioning systems) makes it more appealing to search for techniques that help better understanding of personal thermal comfort level. Since thermal comfort is defined based on the perception of thermal conditions, the current state of the art research is to use user interface for and asking building occupants about their comfort preferences. However, due to fact that these techniques require substantial trainings by the end users, they are not widely used. Intelligence thermostats such as Nest is an example of these techniques.

### SUMMARY

The solution provided by the present system and method is based on the fact that human thermoregulation system adapts to its thermal environment by changing body surface blood vessel diameters. Accordingly the blood flow rates adjust the heat exchange rates with an environment. In case of a cool/cold environment, blood vessels shrink (called vasoconstriction), which results in skin temperature to decrease. In warm/hot conditions, blood vessels widen (called vasodilation), which results in skin temperature to increase. The decrease and increase in skin temperature is not uniform and it varies significantly at different levels of cooling and heating. In order to measure these variations, we have built a glass that is light, cheat and has very accurate infrared sensors attached to it. The first and second prototype (FIG. 8) had components that allowed adjusting sensors locations and enabled us to search for most sensitive points. We will use facial points because (FIG. 9): (1) human face has considerable variations in skin surface blood vessels; (2) facial points are not covered with clothing in office buildings; and most importantly (3) our preliminary experiments on the test subjects show that they are certain points on face that are very good representative of thermoregulation performance.

### BRIEF DESCRIPTION OF DRAWINGS

FIG. 1 illustrates skin blood flow responses to cold stress and heat stress.

FIG. 2 illustrates cutaneous arteries of a male human.

FIG. 3 illustrates three-dimensional vascular territories of all tissues between skin and bone.

FIG. 4 User interface for collecting personal thermal comfort information.

FIGS. 5A-D illustrate glass with installed infrared sensors.

FIG. 6 shows an office space floor plan.

FIG. 7 illustrates data acquisition sensors and receivers formation.

FIG. 8 illustrates two IR sensors mounted on a glass frame.

FIG. 9 illustrates facial points used in a pilot study.

### DETAILED DESCRIPTION

Illustrative embodiments are now discussed and illustrated. Other embodiments may be used in addition or instead. Details which may be apparent or unnecessary may be omitted to save space or for a more effective presentation. Conversely, some embodiments may be practiced without all of the details which are disclosed.

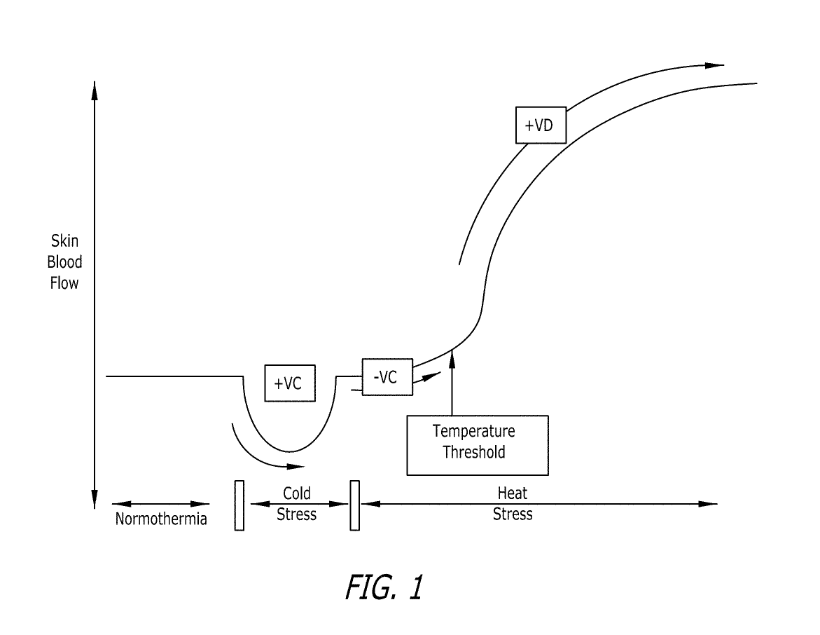

Thermoregulation systems in endothermic animals strive to maintain temperature homeostasis within the body. The temperature homeostasis pertains to the process of regulating internal variables of organs to keep the core body temperature within a range (˜36-38° C.). The brain part that controls thermoregulation system is the preoptic area of the anterior hypothalamus. One of the responses to thermal stresses (i.e., heat and cold) relates to cutaneous vessels. The sympathetic neural control of skin blood flow includes the noradrenergic vasoconstrictor system and cholinergic active vasodilator system. Accordingly, thermoregulation system adjusts the heat dissipation to the external environment by modifying the blood flow via cutaneous arterioles and veins. Resting skin blood flow in the arterioles in normothermic conditions is approximately 250 mL/min (about 5% of cardiac output [151]), which results in a heat dissipation of about 80 to 90 kcal/h (˜the level of resting metabolic heat production). In response to a heat stress, thermoregulatory vasodilation can increase skin blood flow up to 6 to 8 L/min and utilize up to 60% of cardiac output. In response to cold stress, thermoregulatory vasoconstriction can limit the skin blood flow to approach zero. The dual sympathetic neural control mechanisms are performed via two populations of the sympathetic nerves. While non-glabrous skin is covered with both vasoconstrictor and vasodilator nerves, glabrous skin (e.g., palms, soles, and lips) are innervated only by sympathetic vasoconstrictor nerves. Glabrous skin has a rich arteriovenous anastomoses which are thick, low resistance conduits that allow high flow rates directly from arterioles to venules, and are innervated by sympathetic vasoconstrictor nerves. Non-glabrous skin does have a very few arteriovenous anastomoses. The vasoconstrictor system is tonically active in thermonetural environments. Slight changes in the skin blood flow can result in relatively large changes in heat transfer to the environment (an increase in skin blood flow by 8% over the entire body results in doubling the heat transfer to the environment). Solely through changes in cutaneous vasomotor tone, temperature homeostasis can be achieved. Sympathetic vasodilator system is not tonically active in normothermia and is only activated when internal temperature increases (e.g., during exercise or heat exposure). FIG. 1 provides a detailed process of the skin blood flow response to heat and cold stress.

As can be seen in FIG. 1, vasoconstrictor system immediately activates and reduces blood flow during cold stress. After removal of the cold stress, the skin blood flow immediately returns to normothermia conditions. However, vasoconstrictor system can yet help dissipating in heat stress via relaxing the blood vessels to increase the blood flow. Vasodilation system activates when the internal body temperature approaches some threshold.

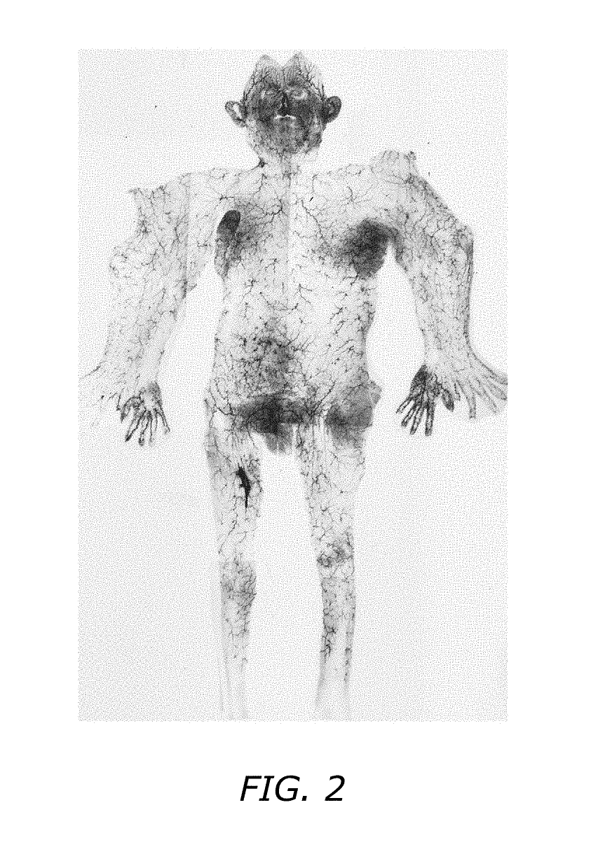

However, the distribution of cutaneous vessels is not uniform across the body (FIG. 2). On areas around the face, the density of vessels is considerably higher.

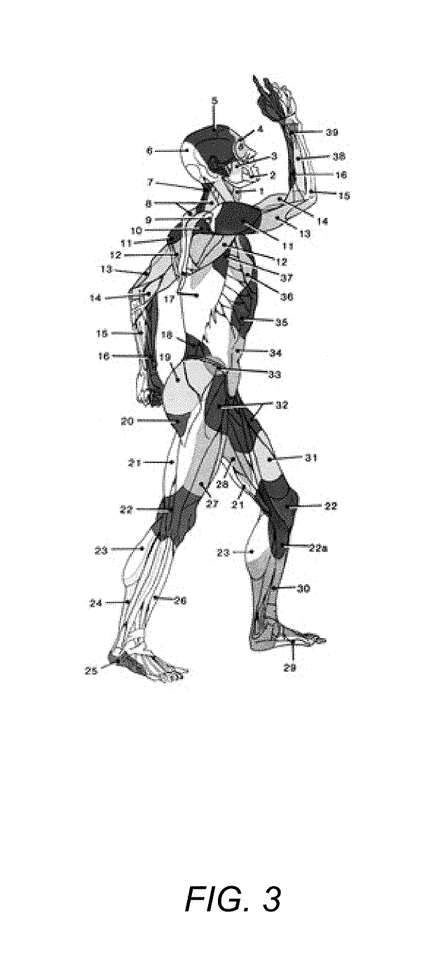

Based on the blood supply to cutaneous vessels and the underlying deep tissues, body can be segregated into three-dimensional vascular territories. The anatomic territories are supplied by a source (segmental or distributing) artery and accompanying veins that span between the skin and the bone. FIG. 3 illustrates the three-dimensional vascular territories of all tissues between skin and bone.

There are several factors that influence the performance of an individual's thermoregulation system: aging, diabetes, vitamins, blood pressure, local cooling and heating, female productive hormones, and cutaneous microvascular disorders.

There are several methods for measuring skin blood flow: venous occlusion plethysmography, laser Doppler blood flow, ultrasound, Thermostrom, Hertzman photoelectric plethysmography, impedance, and radioactive isotopes. However, these methods are not yet studied in an online learning and continuous fashion. In addition, the performance of various cardiovascular territories in response to thermoregulatory actions is not understood.

The methodology of the present system is based on the fact that human face are divided into several territories supplied with different arteries. The thermoregulation system performance as described in the previous section are responses to normothermia, heat stress, cold stress. Accordingly, three states of operations are defined for the present thermoregulation system as (1) neutral state as responses to normothermia, (2) cooling state as responses to heat stress, and (3) heating state as responses to cold stress. In order to map the blood flow from the mentioned territories into the thermoregulation operation states, each vascular territory is first searched separately to find relationships with the thermoregulation operation state. Correlation analysis is then implemented between environment temperature and vascular territories. All the vascular territories are then studied together to learn operation state via use several unsupervised learning algorithms: (1) K-Means, (2) Gaussian Mixture Models (GMM), and (3) Hidden Markov Models (HMM). These learning algorithms are implemented in an online learning manner and continuously update their internal parameters in order to fit the newly introduced input data.

The underlying assumption is that the thermal state is controlled by several thermoregulation systems, each of which functions differently in response to heat or cold stress.

### Testing and Evaluation

In order to evaluate and compare the performance of learning algorithms, room temperatures were collected on four locations around each test subject as the signature of heating and cooling happening the environment. In addition, we designed a web interface in order to collect subjects' thermal comfort. Room temperature represents the external heat and cold source. Thermal comfort is condition of mind and processed by thalamus which is located very closed to anterior hypothalamus (thermoregulation controller). Although both these measures are not the exact representation of thermoregulation system operation state, they can be used as some approximations for evaluation of the learning techniques. Room temperature is representative of the thermal stimulus to the subject. Thermal comfort votes are the conscious perception of thermal environment whereas the unconscious perception of thermal environment is performed by thermoregulation system.

### Data Collection

The data collection was completed in a climate chamber (i.e., an office space) in University of Southern California (USC) campus buildings. Based on the Köppen climate classification, the climate of the area is defined as a dry-summer subtropical climate (also referred to as the Mediterranean climate). For such climates, the average temperature in the warm months is above 10° C. and in the cold months is between −3 and 18° C.

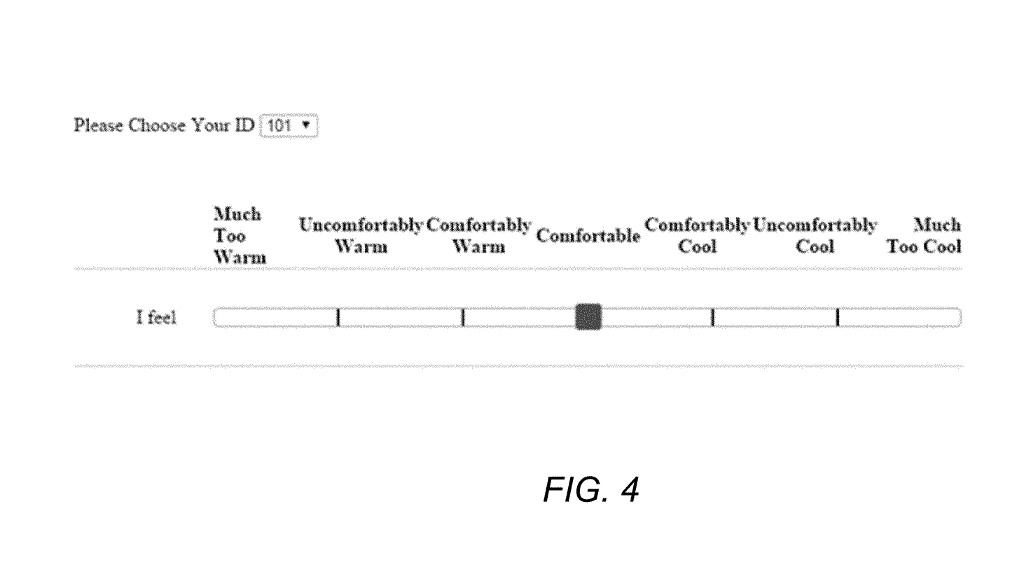

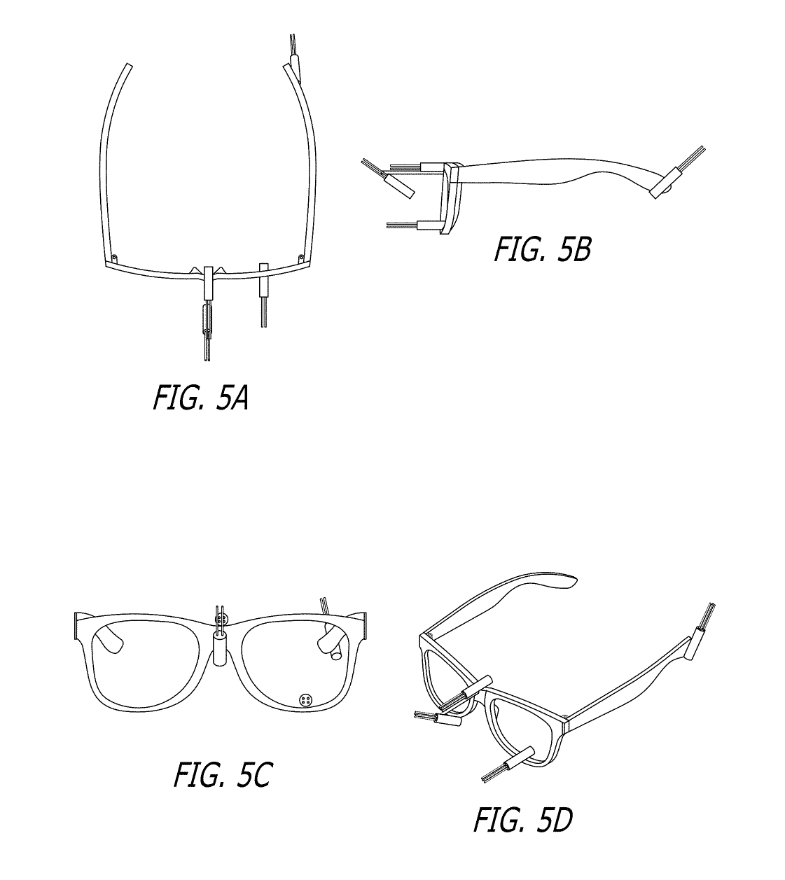

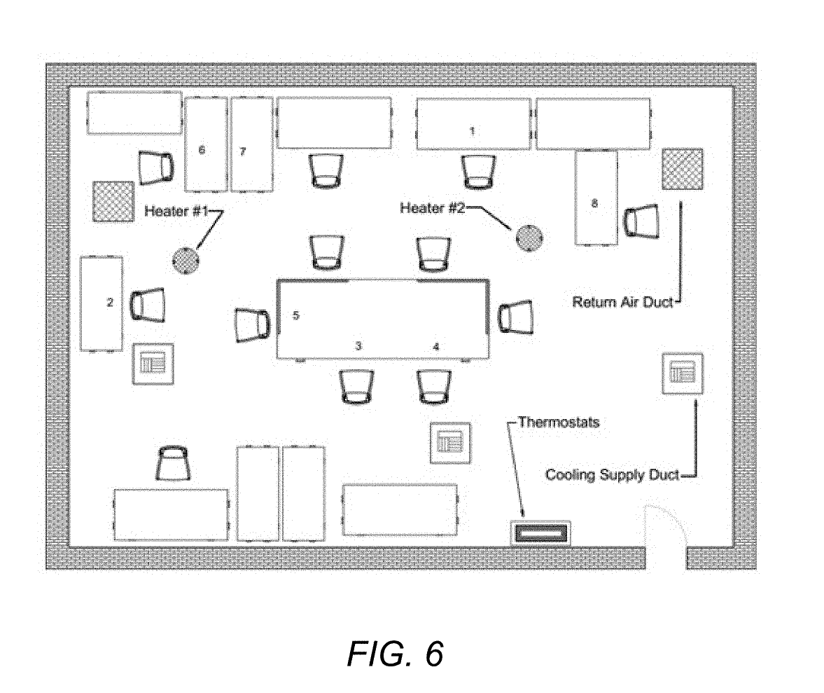

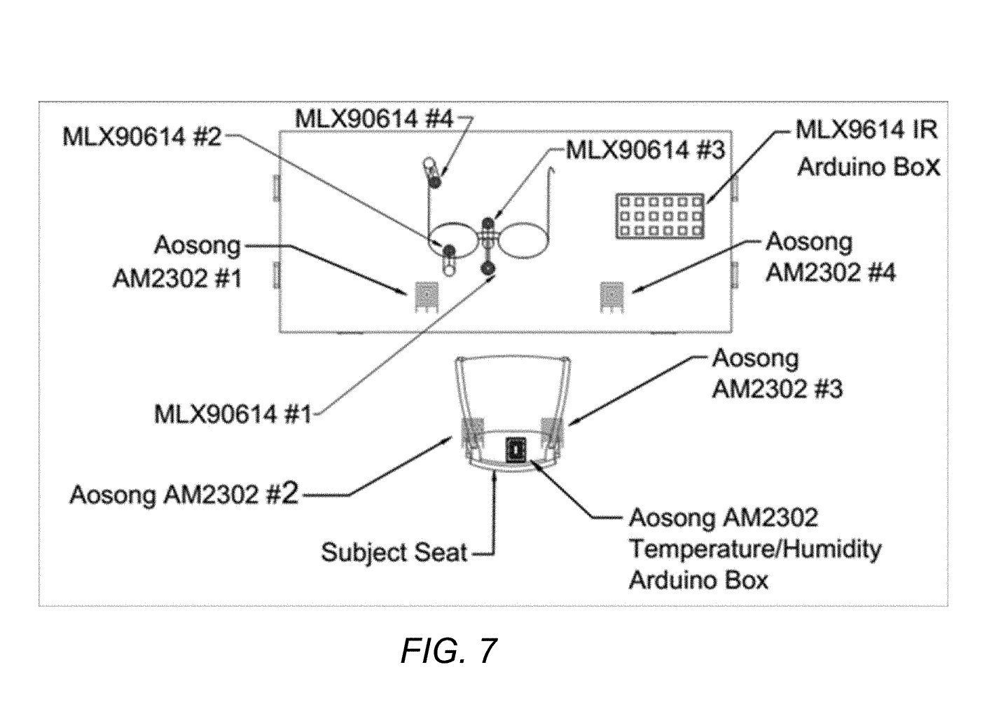

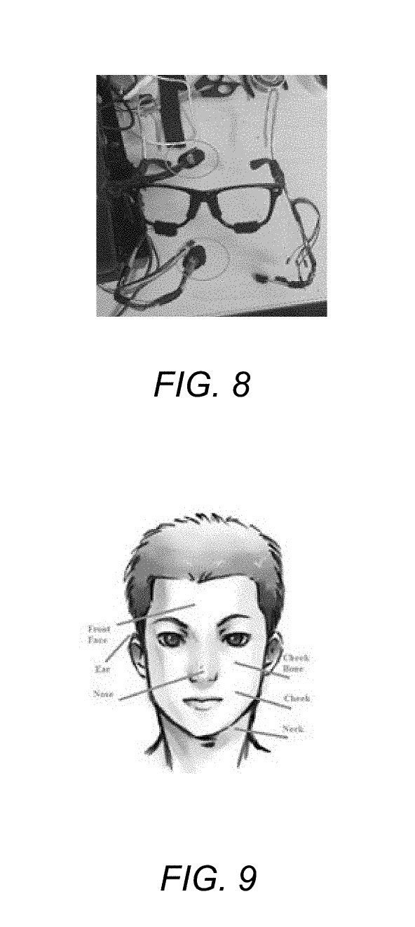

The test subjects included students, staff, and the faculty in the USC campus buildings. Each test subject was given an ID number and asked to communicate his/her votes with that specific ID number, using a user interface (FIG. 4) while wearing a glass with infrared sensors installed on it (FIGS. 5A-D). The test subjects were asked to communicate theft votes while having theft regular office activities in order to be representative of an actual implementation. We also asked the test subjects to communicate a maximum of 10 votes per day. Our goal was to eliminate the bias of the comfort information to a specific day or a condition. Finally, the test subjects were asked not to communicate theft votes very frequently, specifically they were asked to have at least a 15-minute interval between each vote. The plan of the office space is demonstrated in FIG. 6. Numbered tables show the places that subjects were randomly located for the data acquisition. The heating source in the climate chamber was two electrical heaters and the cooling source was the building central air conditioning system controlled with the room thermostat. Different environmental thermal conditions are created through the heating and cooling sources.

Several different machine learning algorithms have been searched for enabling an un-supervised learning of infrared sensors signals. Although thermoregulation operations do not exactly match the brain's perception of thermal comfort, preliminary results have shown that physiological measurements can be used as a heuristic for understanding comfort. This technique may be more applicable to real-world and have higher chances of being used by ordinary people and for medical purposes for the following reasons:

### Advantages

The infrared sensing glass (iGlass) is cheap (sensors cost less than 50$ due to recent advancements in technology) compared to expensive infrared sensing devices.

As long as it is carried by the end user, it continuously monitors the blood vessels shrinking and widening which represents thermoregulation performance. Therefore, uncomfortable/harmful conditions can be detected by supervising the trends in measurements before the conditions actually occur.

It requires no interactions/input from end users, other than turn on/off. This is a huge advantage compared to other techniques.

Humans are adapted to wear glasses. Therefore, wearing the present sensing glass would not be annoying.

It may be possible to integrate the present system into existing systems, such as Google Glass, by adding the sensors to the hardware and the learning algorithms to the software.

The costumers include both companies/organizations such as commercial buildings (to improve the staff productivity and reduce costs by reducing energy consumption), hospitals and fitness centers (to continuously monitor health), and end users who care about their health and home energy consumption costs.

Further details regarding methods, processes, materials, modules, components, steps, embodiments, applications, features, and advantages are set forth in the attached Appendices 1-7, as follows:

Appendix 1: “Monitoring Body Thermoregulation Performance and Estimate Personal Thermal Comfort Via Infrared Thermography” (16 page);

Appendix 2: “A Thermoregulation System and Method” (10 pages);

Appendix 3: “Thermoregulation System Operation State Via iGlass” (2 pages)

Appendix 4: “iGlass Infrared Sensor Glass” (2 pages)

Appendix 5: “QNRF—National Priority Research Program” (35 pages); and

Appendix 6: “Monitoring Worker Fatigue for Improved Site Safety with DAQRI Smart Helmet” (13 pages).

Appendix 7: “Towards Unsupervised Learning of Thermal Comfort Using Infrared Thermography” (21 pages).

The content of Appendices 1-7 is incorporated herein in its entirety. All documents that are cited in Appendices 1-7 are also incorporated herein by reference in their entirety.

The components, steps, features, objects, benefits and advantages which have been discussed are merely illustrative. None of them, nor the discussions relating to them, are intended to limit the scope of protection in any way. Numerous other embodiments are also contemplated. These include embodiments which have fewer, additional, and/or different components, steps, features, objects, benefits and advantages. These also include embodiments in which the components and/or steps are arranged and/or ordered differently.

Unless otherwise stated, all measurements, values, ratings, positions, magnitudes, sizes, and other specifications which are set forth in this specification are approximate, not exact. They are intended to have a reasonable range which is consistent with the functions to which they relate and with what is customary in the art to which they pertain.

### REFERENCES

All articles, patents, patent applications, and other publications which have been cited are hereby incorporated herein by reference.

### Appendix 1

[1] Bureau of Labor Statistics, “Fatal occupational injuries by industry and selected event or exposure, 2014,” 2015.
[2] Bureau of Labor Statistics, “Employer-Reported Workplace Injuries and Illnesses—2014,” 2015.
[3] T. S. Abdelhamid and J. G. Everett, “Physiological Demands during Construction Work,” J. Constr, Eng. Manag., vol. 128, no. 5, pp. 427-437, October 2002.
[4] T. Cheng, G. C. Migliaccio, J. Teizer, and U. C. Gatti, “Data fusion of Real-time Location Sensing and Physiological Status Monitoring for Ergonomics Analysis of Construction Workers,” J. Comput. Civ. Eng., vol. 27, no. 3, pp. 320-335, May 2012.
[5] Härmä, “Individual differences in tolerance to shiftwork: a review,” Ergonomics, vol. 36, no. 1-3, pp. 101-9, January 2007.
[6] G. P. Krueger, “Sustained work, fatigue, sleep loss and performance: A review of the issues,” Work Stress, vol. 3, no. 2, pp. 129-141, April 1989.
[7] N. K. Park, J. Y. Kim, Y. C. Cho, and D. B. Lee, “Relationship Between Fatigue Symptomes and Life Style Factors Among industrial Workers,” Korean J. Occup. Environ. Med., vol. 10, no. 2, pp. 214-226. May 1998.
[8] J. K. Sluiter, E. M. de Croon, T. F. Meijman, and M. H. W. Frings-Dresen, “Need for recovery from work related fatigue and its role in the development and prediction of subjective health complaints,” Occup. Environ. Med., vol. 60, no. >90001, pp. i62-i70, June 2003.
[9] J. D. Ramsey, “Task performance in heat: a review,” Ergonomics, vol. 38, no. 1, pp. 154-65, January 1995.
A. P. C. Chan, M. C. H. Yam, J. W. Y. Chung, and W. Yi, “Developing a heat stress model for construction workers,” J. Facil. Manag., vol. 10, no. 1, pp. 59-74, February 2012.
[11] S. Rowlinson, A. Yunyanjia, B. Li, and C. Chuanjingju, “Management of climatic heat stress risk in construction: a review of practices, methodologies, and future research,” Accid. Anal. Prev., vol. 66, pp. 187-98, May 2014.
[12] U.S. Department of Labor, “Annual summer campaign to prevent heat-related illnesses launched by US Labor Department,” 2014. [Online]. Available: https://www.osha.gov/pls/oshaweb/owadisp.show_document?p_table=NEWS_RELEASES&p_id=26052.
[13] The peninsula—Qatar, “Construction site accidents on the rise,” 2013.
[14] B. Hartmann and A. G. Fleischer, “Physical load exposure at construction sites,” Scand. J. Work. Environ. Health, vol. 31 Supp 2, pp. 88-95, January 2005.
[15] A. P. C. Chan, W. Yi, D. P. Wong, M. C. H. Yam, and D. W. M. Chan, “Determining an optimal recovery time for construction rebar workers after working to exhaustion in a hot and humid environment,” Build. Environ., vol. 58, pp. 163-171, December 2012.
[16] H. Dong, I. Ugaldey, and A. El Saddik, “Development of a fatigue-tracking system for monitoring human body movement,” 2014 IEEE International Instrumentation and Measurement Technology Conference (I2MTC) Proceedings. pp. 786-791, 2014.
[17] R. R. Singh, S. Conjeti, and R. Banerjee, “Biosignal based on-road stress monitoring for automotive drivers,” 2012 National Conference on Communications (NCC). pp. 1-5, 2012.
[18] R. Matthews, N. J. McDonald, P. Hervieux, P. J. Turner, and M. A. Steindorf, “A wearable physiological sensor suite for unobtrusive monitoring of physiological and cognitive state,” Annual International Conference of the IEEE Engineering in Medicine and Biology-Proceedings. pp. 5276-5281, 2007.
[19] P. Sutler and W. Fee, “Fatigue and the Use of Wearable Technology,” in SPE E&P Health, Safety, Security and Environmental Conference-Americas, 2015.
[20] S. Oh, P. S. Kumar, H. Kwon, P. Rai, M. Ramasamy, and V. K. Varadan, “Wireless health monitoring helmet for football players to diagnose concussion and track fatigue,” in SPIE Smart Structures and Materials+Nondestructive Evaluation and Health Monitoring, 2013, vol. 8691, p. 869106.
[21] S. K. L. Lai and A. Craig, “A critical review of the psychophysiology of driver fatigue,” Biol. Psychol., vol. 55, no. 3, pp. 173-194, February 2001.
[22] M. S. Green, Y. Luz, E. Jucha, M. Cocos, and N. Rosenberg, “Factors affecting ambulatory heart rate in industrial workers,” Ergonomics, vol. 29, no. 8, pp. 1017-27, August 1986.
[23] N. Charkoudian, “Skin blood flow in adult human thermoregulation: how it works, when it does not, and why,” Mayo Clin. Proc., vol. 78, no. 5, pp. 603-12, May 2003.
[24] L. B. Rowell, “Cardiovascular Adjustments to Thermal Stress,” in Comprehensive Physiology, John Wiley & Sons, Inc., 2011.
[25] J. M. Johnson and D. W. Proppe, “Cardiovascular adjustments to heat stress,” Compr. Physiol., 2011.
[26] W. L. Kenney and J. M. Johnson, “Control of skin blood flow during exercise,” Med. Sci. Sports Exerc., vol. 24, no. 3, pp. 303-12, March 1992.
[27] C. Huizenga, H. Zhang, E. Arens, and D. Wang, “Skin and core temperature response to partial- and whole-body heating and cooling,” J. Therm. Biol., vol. 29, no. 7-8, pp. 549-558, October 2004.
[28] “Physiology of Thermoregulation.” [Online]. Available: http://intranet.tdmu.edu.ua/data/kafedra/internal/normal_phiz/classes_stud/en/med/lik/2 course/4 Cycle Physiology of breathing/02 Regulation of breathing.htm. [Accessed: 12 Nov. 2015].
[29] L. J. C. van Loon. P. L. Greenhaff, D. Constantin-Teodosiu, W. H. M. Saris, and A. J. M. Wagenmakers, “The effects of increasing exercise intensity on muscle fuel utilisation in humans,” J. Physiol., vol. 536, no. 1, pp. 295-304, October 2001.
[30] G. A. Borg, “Psychophysical bases of perceived exertion,” Med sci Sport. Exerc, vol. 14, no. 5, pp. 377-381,1982.
[31] D. F. Dinges and J. W. Powell, “Microcomputer analyses of performance on a portable, simple visual RT task during sustained operations,” Behav. Res. Methods, Instruments, Comput., vol. 17, no. 6, pp. 652-655, November 1985.
[32] J. Dorrian, G. D. Roach, A. Fletcher, and D. Dawson, “Simulated train driving: fatigue, self-awareness and cognitive disengagement,” Appl. Ergon., vol. 38, no. 2, pp. 155-66, March 2007.
[33] M. Banner' and J. Rubinstein, “Fitness for duty: a 3-minute version of the Psychomotor Vigilance Test predicts fatigue-related declines in luggage-screening performance,” J. Occup. Environ. Med., vol. 53, no. 10, pp. 1146-54, October 2011.
[34] S. A. Ferguson, G. M. Paech, J. Dorrian, G. D. Roach, and S. M. Jay, “Performance on a simple response time task: Is sleep or work more important for miners?,” Appl. Ergon., vol. 42, no. 2, pp. 210-3, January 2011.
[35] J. Geiger-Brown, V. E. Rogers, A. M. Trinkoff, R. L. Kane, R. B. Bausell, and S. M. Scharf, “Sleep, Sleepiness, Fatigue, and Performance of 12-Hour-Shift Nurses,” Chronobiol. Int., February 2012.
[38] S. Li and B. B. Gerber, “Evaluating physiological load of workers with wearable sensors,” Comput. Civ. Eng., no. Cii, pp. 405-412, 2012.

### Appendix 6

[1] Latman, Neal S., et al. “Evaluation of clinical thermometers for accuracy and reliability.” Biomedical instrumentation & technology/Association for the Advancement of Medical Instrumentation 35.4 (2000): 259-265.
[2] Hardy, James D. “Physiology of temperature regulation.” Physiol. Rev 41.521-606 (1961): 221.
[3] Standard, A. S. H. R. A. E. “Standard 55-2004.” Thermal environmental conditions for human occupancy (2004).
[4] R. Z. Freire, G. H. Oliveira, N. Mendes, Predictive controllers for thermal comfort optimization and energy savings, Energy and Buildings. 40 (2008) 1353-1365.
[5] T. Hoyt, K. H. Lee, H. Zhang, E. Arens, T. Webster, Energy savings from extended air temperature setpoints and reductions in room air mixing (2009).
[6] B.E.D. Book, US Department of Energy, 2011 (2010).
[7] https://nest.com/

What is claimed is:

1. A system for learning thermoregulation performance, the system comprising:
one or more infrared sensors operative to detect temperature of the surface of a user's face, and to provide temperature signals indicative of the temperature of the surface of the user's face;
a computer-readable non-transitory storage medium, including computer-readable instructions; and
a processor connected to the memory and operative to receive the temperature signals, wherein the processor, in response to reading the computer-readable instructions, is operative to:
monitor the person's blood vessels shrinking and widening which represents thermoregulation performance; and
detect uncomfortable or harmful conditions by monitoring trends in measurement data before the conditions actually occur.

2. The system of claim 1, wherein the processor is further operative to implement an unsupervised learning algorithm of the temperature signals.
